(19) United States
(12) Patent Application Publication
(10) Pub. No.: US 2026/0144433 A1
(43) Pub. Date: May 28, 2026

(54) RETURN MECHANISMS FOR ENDOSCOPE SUCTION VALVES

(71) Applicant: Boston Scientific Scimed Inc., Maple Grove, MN (US)

(72) Inventors: Kurt Nicholas Robakiewicz, Upton, MA (US); Brian Luis, Boylston, MA (US); Ryan V. Wales, Northborough, MA (US)

(73) Assignee: Boston Scientific Scimed Inc., Maple Grove, MN (US)

(21) Appl. No.: 18/956,840

(22) Filed: Nov. 22, 2024

(51) Int. Cl. A61B 1/015

(52) U.S. Cl. CPC A61B 1/015

(57) ABSTRACT

A suction valve for an endoscope (e.g., a duodenoscope) includes a valve housing, a valve spool, and a return mechanism for returning the valve spool from a manually depressed position to a home position. In the depressed position, the suction valve places the endoscope in a suction configuration for drawing fluids, tissue or other matter out from within a patient. The home position places the endoscope in a vented configuration to interrupt the suction. Various examples of the return mechanism include a resilient integral portion of the valve spool, a resilient integral portion of the valve housing, a resiliently flexible diaphragm extending radially across an annular gap between the valve spool and the valve housing, and a magnetic set affixed to the valve spool and the valve housing.

### CROSS REFERENCE TO RELATED APPLICATIONS

This application claims the benefit of U.S. Provisional Patent Application Ser. No. 63/602,134 filed on Nov. 22, 2023, the disclosure of which is incorporated herein by reference.

### TECHNICAL FIELD

Various aspects of this disclosure relate generally to endoscopes (e.g., duodenoscopes, colonoscopes, bronchoscopes, etc.) and more specifically to endoscope suction valves with means for returning a valve spool to its home position.

### BACKGROUND

Endoscopes enable medical practitioners to directly visualize internal cavities of patients without the need for invasive surgeries. Among the various types of endoscopes, duodenoscopes hold a prominent place due to their capability to explore the upper gastrointestinal tract, particularly the duodenum, pancreas, and bile ducts. Duodenoscopes facilitate not only visual examinations but also a range of therapeutic procedures, making them indispensable tools in modern medicine.

Duodenoscopes typically comprise a flexible tubular probe extending from a handle body. The probe is inserted into the patient, while the operator holds the handle body. A light source at a distal end of the probe provides illumination for viewing. Often a high-resolution camera is adjacent to the light source for capturing real-time images or videos of the internal cavities.

A duodenoscope's probe usually includes an internal working channel, allowing for the insertion of various instruments for procedures like biopsies, tissue removal, or stent placement. To provide maneuverability and access to intricate anatomical structures, many duodenoscope probes have internal wires. The tension in the wires can be adjusted in opposing sets of two by manipulating knobs on a handle body of the duodenoscope. Adjusting the wire tension enables bending and steering of the probe.

Handle bodies typically include a bifluidic valve (air/water valve) and a suction valve, which the operator controls manually. Bifluidic valves can be used for controlling fluid flow, such as air for insufflation and water for irrigation. Insufflation is a technique involving the introduction of air or carbon dioxide into the cavity being examined, which helps to expand the space, allowing for better visibility of the targeted area.

Irrigation typically involves the introduction of liquids, such as sterile water or saline. Irrigation can help clear blood, debris, or mucus from the visual field, ensuring clear visibility. Irrigation can also aid in therapeutic interventions by flushing out areas of interest, allowing for better access and manipulation of tissues.

Suction valves on the handle body are used by the operator to apply or terminate suction at the distal end of the probe. Suction valves often include a valve spool that can be moved manually between open and closed positions. When open, the applied suction can clear the probe's field of view by drawing fluid, tissue and other matter back through a suction tube within the probe. Moving the valve spool to the closed position terminates the suction. Suction valves typically include a separate spring component for urging the valve spool to its closed position.

### SUMMARY

The present disclosure generally pertains to suction valves for use in an endoscope that includes a handle body. In some examples, the suction valve is configurable selectively to a suction configuration and a vented configuration. Some examples of the suction valve include a valve housing supported by the handle body. Some examples of the suction valve include a valve spool that is movable between a home position and a depressed position relative to the valve housing. In some examples, the suction valve is in the suction configuration when the valve spool is in the depressed position. In some examples, the suction valve is in the vented configuration when the valve spool is in the home position. In some examples, the valve spool extends in a lengthwise direction farther into the valve housing when the valve spool is in the depressed position than in the home position. In some examples, the valve housing and/or the valve spool includes both a spring portion and a rigid portion, wherein the spring portion urges the valve spool to the home position.

In some examples, the suction valve includes a valve housing supported by the handle body. Some examples of the suction valve include a diaphragm with an outer periphery and an inner periphery. In some examples, the outer periphery is anchored to the valve housing and/or the handle body. In some examples, the inner periphery defines an aperture. Some examples of the suction valve include a valve spool engaging the inner periphery of the diaphragm. In some examples, the valve spool extends in a lengthwise direction through the aperture and into the valve housing. In some examples, the valve spool is movable between a home position and a depressed position relative to the valve housing. In some examples, the suction valve is in the suction configuration when the valve spool is in the depressed position. In some examples, the suction valve is in the vented configuration when the valve spool is in the home position. In some examples, the valve spool extends farther into the valve housing when the valve spool is in the depressed position than in the home position. In some examples, the diaphragm is resiliently flexible to urge the valve spool to the home position.

In some examples, the suction valve includes a valve housing supported by the handle body. Some examples of the suction valve include a valve spool being movable between a home position and a depressed position relative to the valve housing. In some examples, the suction valve is in the suction configuration when the valve spool is in the depressed position. In some examples, the suction valve is in the vented configuration when the valve spool is in the home position. In some examples, the valve spool extends in a lengthwise direction farther into the valve housing when the valve spool is in the depressed position than in the home position. Some examples of the suction valve include a magnetic set comprising a first member substantially stationary relative to the valve spool and a second member substantially stationary relative to the valve housing. In some examples, the magnetic set provides a magnetic force urging the valve spool to the home position

The preceding summary is provided to facilitate an understanding of some of the features of the present disclosure and is not intended to be a full description. A full appreciation of the disclosure can be gained by taking the entire specification, claims, drawings and abstract as a whole.

### BRIEF DESCRIPTION OF THE DRAWINGS

FIG. 1 is a perspective view of an example endoscope with a suction valve incorporating various aspects of the present disclosure.

FIG. 2 is a perspective view schematically illustrating an example suction valve for the endoscope shown in FIG. 1.

FIG. 3 is a cross-sectional view taken along line 3-3 of FIG. 2.

FIG. 4 is a cross-sectional view similar to FIG. 3 but showing another example suction valve.

FIG. 5 is a front view of an example valve spool on the left and a front cross sectional view of an example valve housing on the right.

FIG. 6 is a cross-sectional view taken along line 6-6 of FIG. 5.

FIG. 7 is a cross-sectional view taken along line 7-7 of FIG. 5.

FIG. 8 is a cross-sectional view similar to FIG. 6 but of another example valve spool.

FIG. 9 is a cross-sectional view similar to FIG. 7 but of the same example valve spool as in FIG. 8.

FIG. 10 is a front view of an example valve spool inserted in an example valve housing shown in cross-section, wherein the suction valve is in a vented configuration.

FIG. 11 is a front view similar to FIG. 10 but showing the suction valve in a suction configuration.

FIG. 12 is a front assembly view of an example suction valve.

FIG. 13 is a cross-sectional view taken along line 13-13 of FIG. 12.

FIG. 14 is a cross-sectional view taken along line 14-14 of FIG. 12.

FIG. 15 is a cross-sectional view similar to FIG. 13 but of another example valve spool.

FIG. 16 is a cross-sectional view similar to FIG. 14 but of the same example valve spool as in FIG. 15.

FIG. 17 is a front view of an example suction valve in a vented configuration.

FIG. 18 is a front view similar to FIG. 17 but showing the suction valve in a suction configuration.

FIG. 19 is a front view of an example valve spool inserted in an example valve housing shown in cross-section, wherein the suction valve is in a vented configuration.

FIG. 20 is a front view similar to FIG. 19 but showing the suction valve in a suction configuration.

FIG. 21 is a front view of an example suction valve in a vented configuration.

FIG. 22 is a front view similar to FIG. 21 but showing the suction valve in a suction configuration.

FIG. 23 is a front view of an example valve spool inserted in an example valve housing shown in cross-section, wherein the suction valve is in a vented configuration.

FIG. 24 is a front view similar to FIG. 23 but showing the suction valve in a suction configuration.

FIG. 25 is a front view of an example suction valve in a vented configuration.

FIG. 26 is a front view similar to FIG. 25 but showing the suction valve in a suction configuration.

FIG. 27 is a front view of an example valve spool on the left and a front cross sectional view of an example valve housing on the right.

FIG. 28 is a front view of an example valve spool inserted in an example valve housing shown in cross-section, wherein the suction valve is in a vented configuration.

FIG. 29 is a front view similar to FIG. 28 but showing the suction valve in a suction configuration.

FIG. 30 is a front view of an example valve spool inserted in an example valve housing shown in cross-section, wherein the suction valve is in a vented configuration.

FIG. 31 is a front view similar to FIG. 30 but showing the suction valve in a suction configuration.

FIG. 32 is a cross-sectional view taken along line 32-32 of FIG. 30.

FIG. 33 is a front view of an example valve spool inserted in an example valve housing shown in cross-section, wherein the suction valve is in a vented configuration.

FIG. 34 is a front view similar to FIG. 33 but showing the suction valve in a suction configuration.

FIG. 35 is a front view of an example valve spool inserted in an example valve housing shown in cross-section, wherein the suction valve is in a vented configuration.

FIG. 36 is a front view similar to FIG. 35 but showing the suction valve in a suction configuration.

FIG. 37 is a front view of an example valve spool inserted in an example valve housing shown in cross-section, wherein the suction valve is in a vented configuration.

FIG. 38 is a front view similar to FIG. 37 but showing the suction valve in a suction configuration.

FIG. 39 is a front view of an example valve spool inserted in an example valve housing shown in cross-section, wherein the suction valve is in a vented configuration.

FIG. 40 is a front view similar to FIG. 39 but showing the suction valve in a suction configuration.

### DESCRIPTION

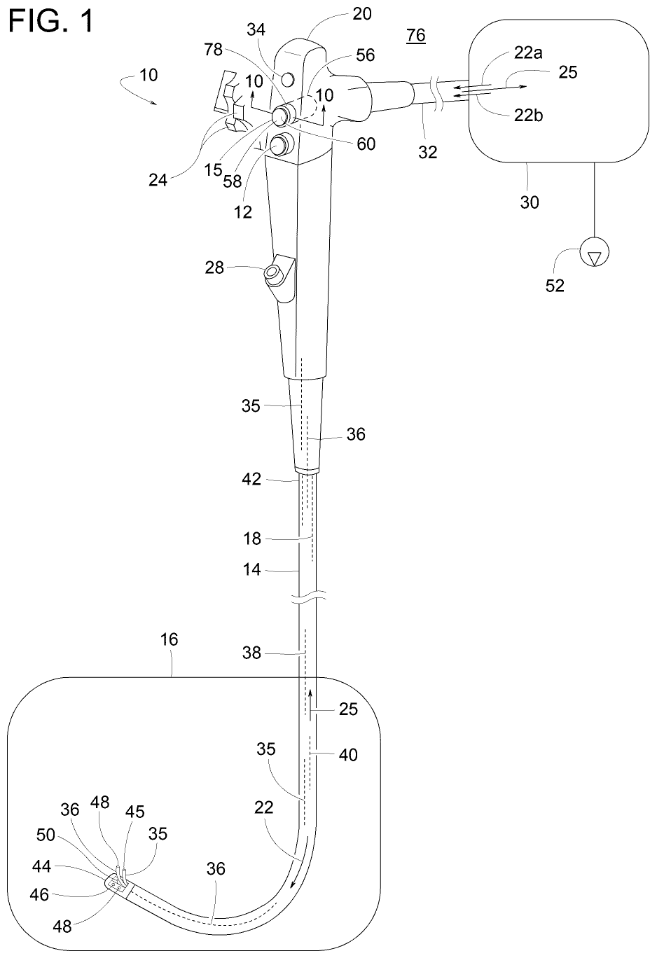

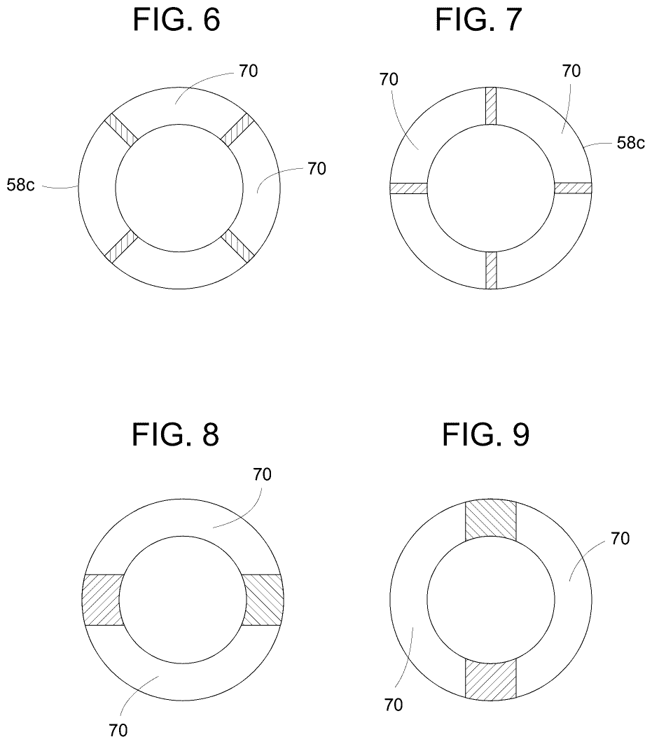

FIGS. 1-40 show various examples of a suction valve 15 (e.g., suction valves 15a-m) for an endoscope 10 and methods for using them. The term, “endoscope” represents any medical apparatus with a flexible tubular probe 14 for inserting into a patient 16 to visually explore the patient's internal tissues and cavities and to introduce or withdraw water, air, or other fluids when desired. Some example endoscopes 10 have internal wires 18 with adjustable tension for bending and steering the flexible tubular probe 14. Some examples of the endoscope 10, shown in FIG. 1, include duodenoscopes, colonoscopes, ureteroscopes, bronchoscopes, laparoscopes, sheaths, and catheters.

The endoscope 10 is illustrated as an example, so many of the following listed components are optional. Some examples of the endoscope 10 include components such as a handle body 20, the flexible tubular probe 14 extending from the handle body 20, the suction valve 15 for controlling the suction of a fluid 25 (e.g., bodily fluids) drawn back through the flexible tubular probe 14, a bifluidic valve 12 (air/water valve) for controlling the flow of a fluid 22 (e.g., a liquid 22a and a gas 22b), steering knobs 24 to adjust the tension in the internal wires 18, a biopsy port 28 for sampling withdrawn tissue or fluid, a control unit 30, an umbilicus 32 connecting the control unit 30 to the handle body 20, and an image capture button 34.

The control unit 30 and/or its associated external components provide various functions, such as supplying liquid 22a (e.g., water, saline, etc.), supplying gas 22b (e.g., air, carbon dioxide, etc.), providing a source of vacuum 52 (e.g., a vacuum pump, venturi, etc.), sending and receiving electrical signals, processing electrical signals, etc. Some of these functions are optional. The umbilicus 32 connects the control unit 30 in signal communication or fluid communication with the bifluidic valve 12, the suction valve 15, the flexible tubular probe 14, or other endoscope-related components. The term, “vacuum” means anything less than atmospheric pressure (i.e., less than 14.7 psia).

In some examples, the flexible tubular probe 14 contains various components such as the internal wires 18 for steering, tubing 36 (one or more tubes) for conveying fluids 22, a suction tube 35 for drawing the fluid 25 from the patient 16, a fiber optic cable 38 for conveying images or light, and electrical wires 40 for conveying electrical power or signals. Some of these probe components are optional.

The flexible tubular probe 14 has a proximal end 42 and a distal end 44. The proximal end 42 connects to the handle body 20, and the distal end 44 extends away from the handle body 20. At the distal end 44, some examples of the flexible tubular probe 14 include a light 46 (or fiber optic cable leading thereto) for illuminating a patient's internal cavities, a camera 47 (or fiber optic cable leading thereto), a tip 45 of the suction tube 35, a tip 48 of the tubing 36, and an elevator 50 for tilting tips 45 and/or 48. The elevator 50 is also known as a swing stand, a pivot stand, and a raising bed.

The tip 48 of the tubing 36 is open to deliver fluid 22 to the patient 16 for insufflating or irrigating. The tip 45 of the suction tube 35 is open to draw fluids 25 from within the patient 16. In some examples, the source of vacuum 52 draws the fluid 25 in series from the patient 16, through the open tip 45, through the suction tube 35, through the suction valve 15, through the umbilicus 32, and out through the control unit 30.

The suction valve 15 determines whether suction is applied to the suction tube 35. In some examples, the suction valve 15 includes a valve housing 56 (e.g., valve housings 56a-m) and a valve spool 58 (e.g., valve spools 58a-m) with a pushbutton head 60. Manually depressing the pushbutton head 60 moves the valve spool 58 farther into the valve housing 56 to deliver suction to the suction tube 35. Releasing the pushbutton head 60 returns the valve spool 58 to its home position, discontinuing the suction to the suction tube 35.

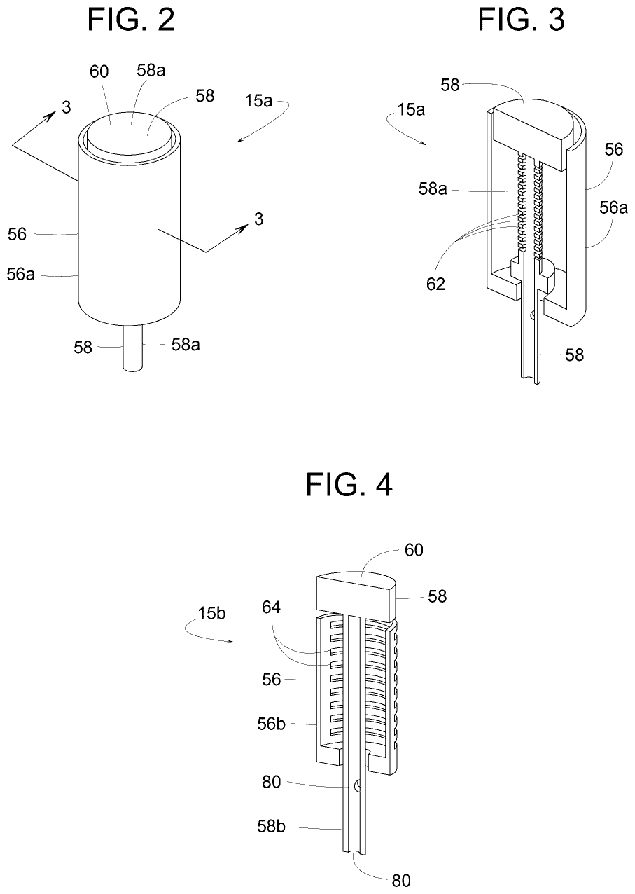

FIGS. 2-4 schematically illustrate two concepts for returning the valve spool 58 to its home position. In the example shown in FIGS. 2 and 3, the suction valve 15a comprises the valve housing 56a and the valve spool 58a. The valve spool 58a includes a plurality of slits 62 that make it resiliently compressible. The resilient compressibility of the valve spool 58a urges the valve spool 58a to its home position. In the example shown in FIG. 4, the suction valve 15b comprises the valve housing 56b and the valve spool 58b. The valve housing 56b includes a plurality of slits 64 that make it resiliently compressible. The resilient compressibility of the valve housing 56b urges the valve spool 58b to its home position. The basic concepts taught in FIGS. 2-4 can also be applied to the various examples shown in FIGS. 1, 5-40.

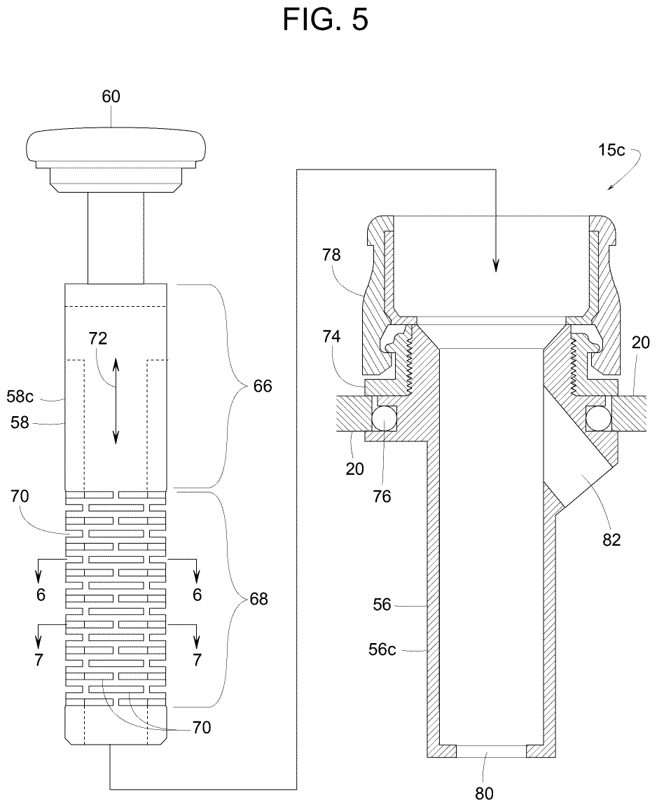

FIGS. 5-11 show suction valve 15c comprising the valve housing 56c and the valve spool 58c. FIG. 5 shows the valve spool 58c being readily inserted into the valve housing 56c with no tools required. In this example, valve spool 58c comprises a rigid portion 66 and a spring portion 68. The terms, “spring portion” and “rigid portion” refer to the relative flexibility of the two portions, rather than their absolute flexibility. A spring portion or a rigid portion does not imply any specific degree of flexibility, structure or geometry. Rather, the spring portion is simply more flexible than the rigid portion, and the rigid portion is stiffer than the spring portion. In some examples, the spring portion 68 and the rigid portion 66 comprise a seamless unitary piece.

In the example of suction valve 15c, the spring portion 68 has a plurality of slits 70 extending at least partially around the valve spool 58c, providing the spring portion 68 with resilient compressibility. The slits 70 are distributed and spaced apart along the valve spool's lengthwise direction 72 (axial direction). In some examples, the slits 70 are aligned one directly above the other (e.g., slits 62 in FIG. 3). In the example of valve spool 58c, however, the slits 72 are distributed circumferentially and staggered from one layer to the next, as shown in FIGS. 5-7. In some examples, the circumferentially staggered arrangement can provide more axial compression in a manner similar to that of a known wave spring. In some examples, the slits 72 are distributed as shown in FIGS. 8 and 9 for simplicity and ease of manufacturing with conventional tooling.

Some examples of the suction valve 15 include a threaded collar 74 for securing the valve housing 56 to the handle body 20. In some examples, an O-ring 76 seals the valve housing 56 to the handle body 20. In some examples, a button guard 78 encircles the valve spool 58 and snaps onto the collar 74 to anchor the button guard 78 to the handle body 20. The button guard 78 helps prevent the pushbutton head 60 from getting snagged or accidentally depressed.

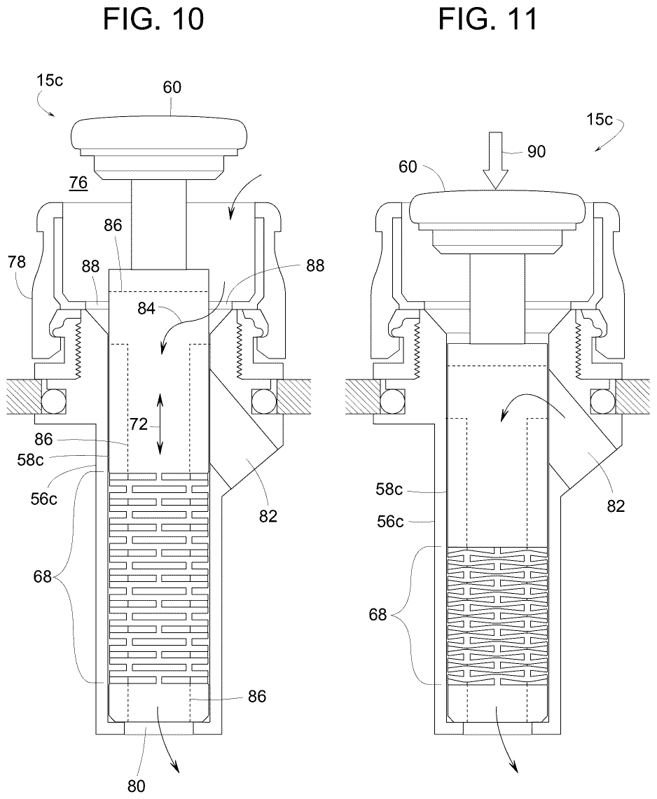

Referring to FIGS. 10 and 11, some examples of the suction valve 15 (15c) include a suction source opening 80 at least partially defined by the valve housing 56 (56c) and/or the valve spool 58 (58c) and being connected in fluid communication with the source of vacuum 52, a probe port 82 at least partially defined by the valve housing 56c to connect the suction valve 15c in fluid communication with the suction tube 35, a vent pathway 84 at least partially defined by the valve housing 56c and/or the valve spool 58c, and a valve spool passageway 86 at least partially defined by the valve spool 58c. In some examples, the button guard 78 and the valve spool 58c define an annular gap 88 therebetween, wherein the annular gap 88 is part of the vent pathway 84. The valve spool passageway 86 is connected in fluid communication with the suction source opening 80.

In some examples, the suction source opening 80 and the pushbutton head 60 are at opposite ends of the valve spool 58c to make the suction valve 15c readily installable within the handle body 20 of the endoscope 10. In some examples, the spring portion 68 is closer to the suction source opening 80 than to the pushbutton head 60 to make the suction valve 15c readily adaptable for use in the endoscope 10. In some examples, the probe port 82 is at an intermediate position between the pushbutton head 60 and the suction source opening 80, wherein the intermediate position is with reference to the lengthwise direction 72. The intermediate position works well for fitting the suction valve 15c, the valve housing 56c and the valve spool 58c within the handle body 20.

Moving valve spool 58 from a home position to a depressed position changes the suction valve's configuration from a vented configuration to a suction configuration. FIG. 10 shows the valve spool 58c at its home position to place the suction valve 15c in the vented configuration. FIG. 11 shows valve spool 58c at its depressed position, placing suction valve 15c in the suction configuration.

In the vented configuration, shown in FIG. 10, the spring portion 68 of the valve spool 58c is relaxed and extended. In this position, the valve spool 58c opens the vent pathway 84 and blocks off the probe port 82. With the vent pathway 84 open, atmospheric air 76 flows freely into the valve spool passageway 86 to negate the vacuum that might otherwise exist in the valve spool passageway 86. Blocking the probe port 82 and venting the valve spool passageway 86 inhibits vacuum from being applied to the suction tube 35 in the flexible tubular probe 14.

Manually depressing 90 the valve spool 58c, as shown in FIG. 11, resiliently compresses the spring portion 68 of the valve spool 58c. In this position, the valve spool 58c opens the probe port 82 and closes the vent pathway 84. With the vent pathway 84 closed, atmospheric air 76 can no longer flow into the valve spool passageway 86, so the source of vacuum 52 can restore the vacuum in the valve spool passageway 86. The now open probe port 82 connects the vacuum in the valve spool passageway 86 to the suction tube 35 in the flexible tubular probe 14, delivering suction to the tip 45 of the suction tube 35.

Upon manually releasing the pushbutton head 60, the resiliently compressed spring portion 68 relaxes and extends. This returns the valve spool 58c to its home position and the suction valve 15c to its vented configuration.

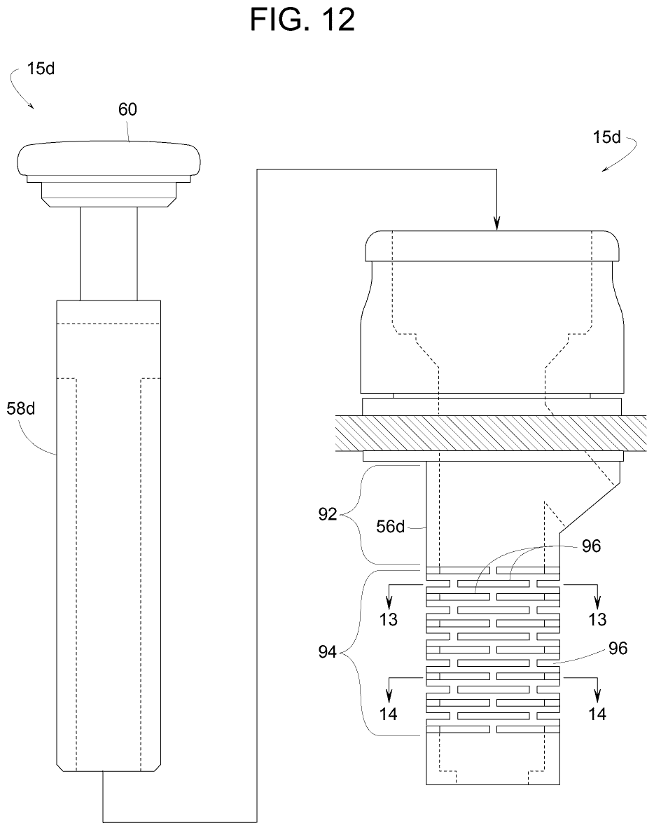

FIGS. 12-18 show suction valve 15d comprising the valve housing 56d and the valve spool 58d. In this example, valve housing 56d comprises a rigid portion 92 and a spring portion 94. In some examples, the spring portion 94 and the rigid portion 92 comprise a seamless unitary piece.

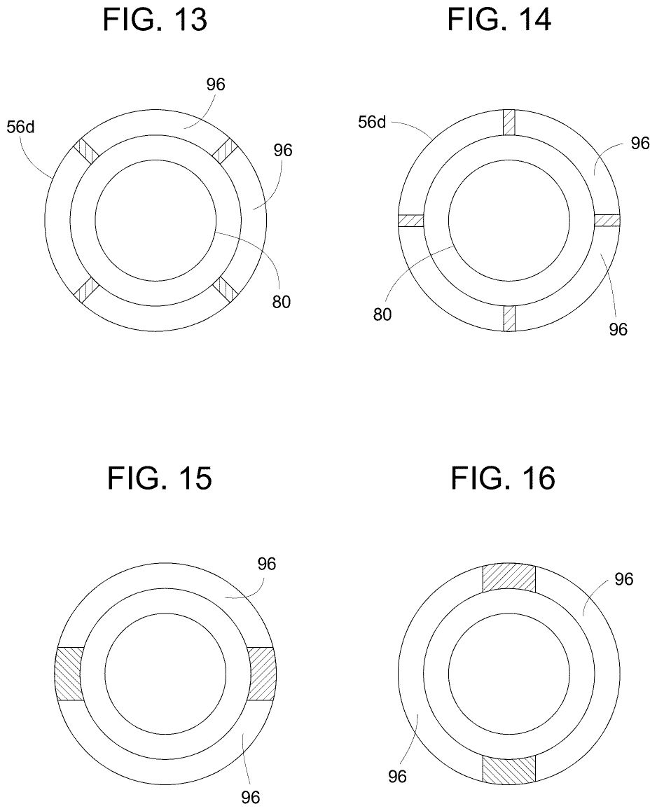

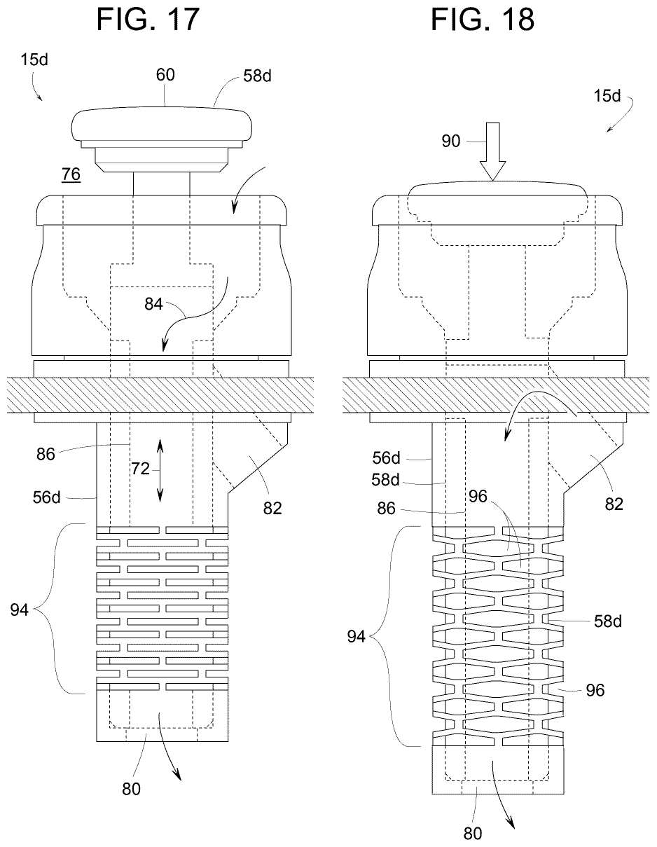

The spring portion 94 of the valve housing 56d has a plurality of slits 96. When the valve spool 58d is inserted in the valve housing 56d, as shown in FIGS. 17 and 18, the slits 96 in the valve housing 56d run at least partially around the valve spool 58d (when installed), providing the spring portion 94 with resilient stretchability. The slits 96 are distributed and spaced apart along the valve housing 56d in the lengthwise direction 72 (axial direction defined by the valve spool). In some examples, the slits 96 are aligned one directly above the other, as shown in FIG. 3. In the example of valve spool 58d, however, the slits 96 are distributed circumferentially and staggered from one layer to the next, as shown in FIGS. 12-14 . In some examples, the circumferentially staggered arrangement can provide more axial stretch. In some examples, the slits 96 are distributed as shown in FIGS. 15 and 16 for simplicity and ease of manufacturing with conventional tooling.

Moving the valve spool 58d from a home position to a depressed position changes the suction valve's configuration from a vented configuration to a suction configuration. FIG. 17 shows the valve spool 58d at its home position to place suction valve 15d in the vented configuration. FIG. 18 shows valve spool 58d at its depressed position, placing suction valve 15d in the suction configuration.

In the vented configuration, shown in FIG. 17, the spring portion 94 of the valve housing 56d is relaxed and retracted. In this position, the valve spool 58d opens the vent pathway 84 and blocks off the probe port 82. With the vent pathway 84 open, atmospheric air 76 flows freely into the valve spool passageway 86 to negate the vacuum that might otherwise exist in the valve spool passageway 86. Blocking the probe port 82 and venting the valve spool passageway 86 inhibits vacuum from being applied to the suction tube 35 in the flexible tubular probe 14.

Manually depressing 90 the valve spool 58d, as shown in FIG. 18, resiliently stretches the spring portion 94 of the valve housing 56d. In this position, the valve spool 58c opens the probe port 82 and closes the vent pathway 84. With the vent pathway 84 closed, atmospheric air 76 can no longer flow into the valve spool passageway 86, so the source of vacuum 52 can restore the vacuum in the valve spool passageway 86. The now open probe port 82 connects the vacuum in the valve spool passageway 86 to the suction tube 35 in the flexible tubular probe 14, delivering suction to the tip 45 of the suction tube 35.

Upon manually releasing the pushbutton head 60, the resiliently stretched spring portion 94 relaxes and retracts. This returns the valve spool 58c to its home position and the suction valve 15d to its vented configuration.

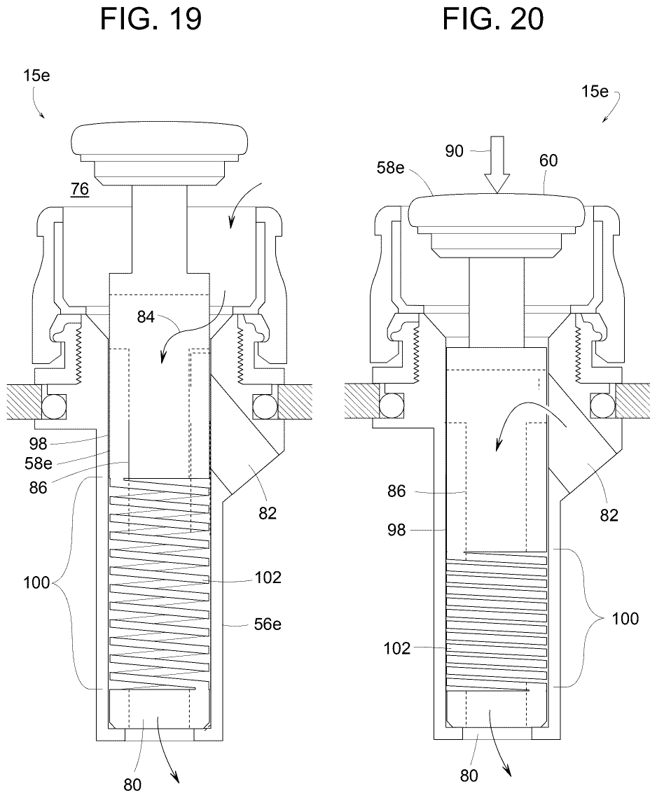

FIGS. 19 and 20 show suction valve 15e comprising the valve housing 56e and the valve spool 58e. The valve spool 58e comprises a rigid portion 98 and a spring portion 100. In some examples, the spring portion 100 is a helical coil 102 extending integrally and seamlessly from the rigid portion 98, whereby the spring portion 100 and the rigid portion 98 comprise a seamless unitary piece. The spring portion 102 provides the valve spool 58e with resilient compressibility.

Moving valve spool 58e from a home position to a depressed position changes the suction valve's configuration from a vented configuration to a suction configuration. FIG. 19 shows the valve spool 58e at its home position to place suction valve 15e in the vented configuration. FIG. 20 shows valve spool 58e at its depressed position, placing suction valve 15e in the suction configuration.

In the vented configuration, shown in FIG. 19, the spring portion 100 of the valve spool 58e is relaxed and extended. In this position, the valve spool 58e opens the vent pathway 84 and blocks off the probe port 82. With the vent pathway 84 open, atmospheric air 76 flows freely into the valve spool passageway 86 to negate the vacuum that might otherwise exist in the valve spool passageway 86. Blocking the probe port 82 and venting the valve spool passageway 86 inhibits vacuum from being applied to the suction tube 35 in the flexible tubular probe 14.

Manually depressing 90 the valve spool 58e, as shown in FIG. 20, resiliently compresses the spring portion 102 of the valve spool 58e. In this position, the valve spool 58e opens the probe port 82 and closes the vent pathway 84. With the vent pathway 84 closed, atmospheric air 76 can no longer flow into the valve spool passageway 86, so the source of vacuum 52 can restore the vacuum in the valve spool passageway 86. The now open probe port 82 connects the vacuum in the valve spool passageway 86 to the suction tube 35 in the flexible tubular probe 14, delivering suction to the tip 45 of the suction tube 35.

Upon manually releasing the pushbutton head 60, the resiliently compressed spring portion 100 relaxes and extends. This returns the valve spool 58e to its home position and the suction valve 15e to its vented configuration.

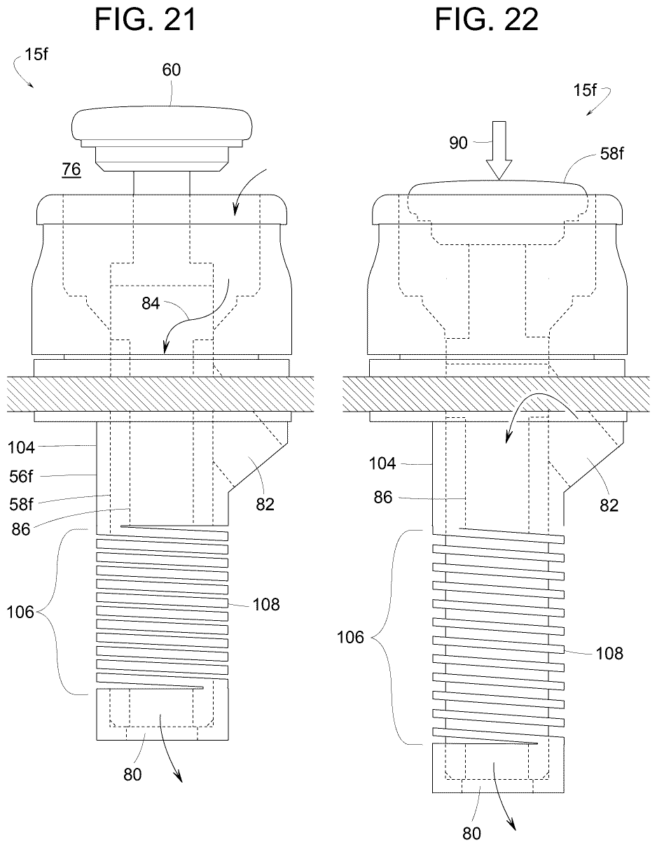

FIGS. 21 and 22 show suction valve 15f comprising the valve housing 56f and the valve spool 58f. The valve housing 56f comprises a rigid portion 104 and a spring portion 106. In this example, the spring portion 106 is a helical coil 108 extending integrally and seamlessly from the rigid portion 104, whereby the spring portion 106 and the rigid portion 104 comprise a seamless unitary piece. The spring portion 106 provides the valve housing 56f with resilient stretchability.

Moving valve spool 58f from a home position to a depressed position changes the suction valve's configuration from a vented configuration to a suction configuration. FIG. 21 shows the valve spool 58f at its home position to place suction valve 15f in the vented configuration. FIG. 22 shows valve spool 58f at its depressed position, placing suction valve 15f in the suction configuration.

In the vented configuration, shown in FIG. 21, the spring portion 106 of the valve housing 56f is relaxed and retracted. In this position, the valve spool 58f opens the vent pathway 84 and blocks off the probe port 82. With the vent pathway 84 open, atmospheric air 76 flows freely into the valve spool passageway 86 to negate the vacuum that might otherwise exist in the valve spool passageway 86. Blocking the probe port 82 and venting the valve spool passageway 86 inhibits vacuum from being applied to the suction tube 35 in the flexible tubular probe 14.

Manually depressing 90 the valve spool 58f, as shown in FIG. 22, resiliently stretches the spring portion 106 of the valve housing 56f. In this configuration, the valve spool 58f opens the probe port 82 and closes the vent pathway 84. With the vent pathway 84 closed, atmospheric air 76 can no longer flow into the valve spool passageway 86, so the source of vacuum 52 can restore the vacuum in the valve spool passageway 86. The now open probe port 82 connects the vacuum in the valve spool passageway 86 to the suction tube 35 in the flexible tubular probe 14, delivering suction to the tip 45 of the suction tube 35.

Upon manually releasing the pushbutton head 60, the resiliently stretched spring portion 106 relaxes and retracts. This returns the valve spool 58f to its home position and the suction valve 15f to its vented configuration.

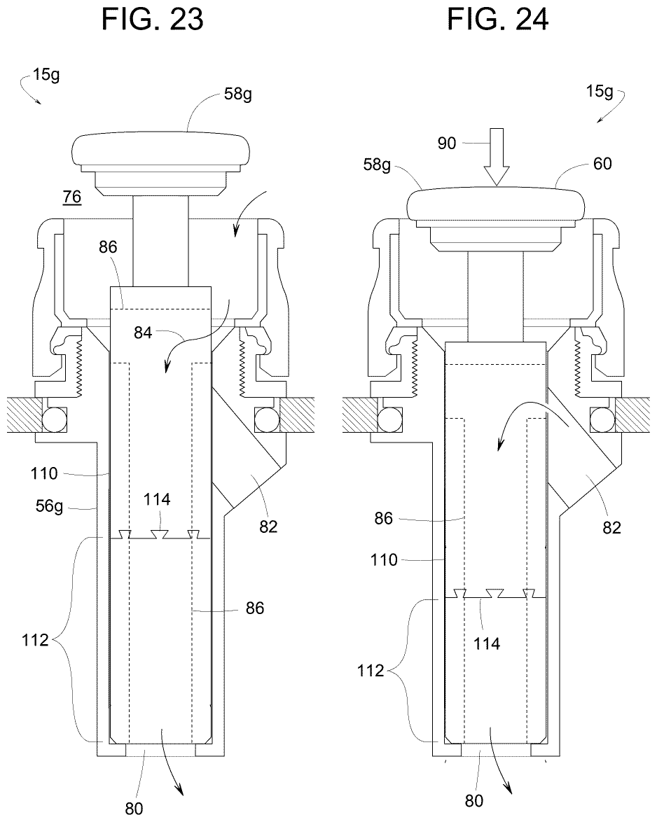

FIGS. 23 and 24 show suction valve 15g comprising the valve housing 56g and the valve spool 58g. The valve spool 58g comprises a rigid portion 110 and a spring portion 112. In some examples, both portions 110 and 112 are made of a polymer for ease of manufacturing or minimizing friction within the valve housing 56g. In some examples, the spring portion 112 is softer and more resiliently compressible than the rigid portion 110. In some examples, the spring portion 112 is comprised of an elastomer (e.g., TPE or thermoplastic elastomer, silicone, polyurethane, neoprene, natural rubber, latex, synthetic rubber, elastane, etc.). The term, “elastomer” refers to any resiliently stretchable or resiliently compressible polymer. In some examples, the rigid portion 110 and the spring portion 112 are co-molded or overmolded together to create a single structure that, in some examples, includes a secure seam 114 between the two portions 110 and 112.

Moving valve spool 58g from a home position to a depressed position changes the suction valve's configuration from a vented configuration to a suction configuration. FIG. 23 shows the valve spool 58g at its home position to place suction valve 15g in the vented configuration. FIG. 24 shows valve spool 58g at its depressed position, placing suction valve 15g in the suction configuration.

In the vented configuration, shown in FIG. 23, the spring portion 112 of the valve spool 58g is relaxed and extended. In this position, the valve spool 58g opens the vent pathway 84 and blocks off the probe port 82. With the vent pathway 84 open, atmospheric air 76 flows freely into the valve spool passageway 86 to negate the vacuum that might otherwise exist in the valve spool passageway 86. Blocking the probe port 82 and venting the valve spool passageway 84 inhibits vacuum from being applied to the suction tube 35 in the flexible tubular probe 14.

Manually depressing 90 the valve spool 58g, as shown in FIG. 24, resiliently compresses the spring portion 112 of the valve spool 58g. In this position, the valve spool 58g opens the probe port 82 and closes the vent pathway 84. With the vent pathway 84 closed, atmospheric air 76 can no longer flow into the valve spool passageway 86, so the source of vacuum 52 can restore the vacuum in the valve spool passageway 86. The now open probe port 82 connects the vacuum in the valve spool passageway 86 to the suction tube 35 in the flexible tubular probe 14, delivering suction to the tip 45 of the suction tube 35.

In some examples, the suction valve 15g has extra radial clearance between the outer diameter of the valve spool 58g and the inner diameter of the valve housing 56g. The extra clearance provides room for the spring portion 112 to expand radially outward when compressed.

Upon manually releasing the pushbutton head 60, the resiliently compressed spring portion 112 relaxes and extends. This returns the valve spool 58g to its home position and the suction valve 15g to its vented configuration.

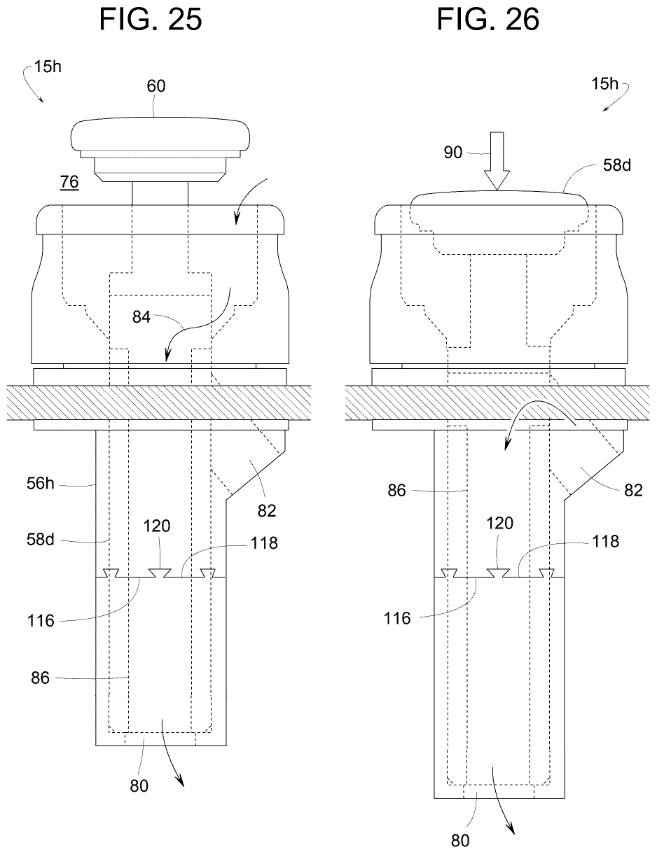

FIGS. 25 and 26 show suction valve 15h comprising the valve housing 56h and the valve spool 58h. The valve housing 56h comprises a rigid portion 116 and a spring portion 118. In some examples, both portions 116 and 118 are made of a polymer for ease of manufacturing or minimizing friction. In some examples, the spring portion 118 is softer and more resiliently stretchable than the rigid portion 116. In some examples, the spring portion 118 is comprised of an elastomer (e.g., TPE or thermoplastic elastomer, silicone, polyurethane, etc.). In some examples, the rigid portion 116 and the spring portion 118 are co-molded or overmolded together to create a single structure that, in some examples, includes a secure seam 120 between the two portions 116 and 118.

Moving valve spool 58h from a home position to a depressed position changes the suction valve's configuration from a vented configuration to a suction configuration. FIG. 25 shows the valve spool 58h at its home position to place suction valve 15h in the vented configuration. FIG. 26 shows valve spool 58h at its depressed position, placing suction valve 15h in the suction configuration.

In the vented configuration, shown in FIG. 25, the spring portion 118 of the valve spool 58h is relaxed and extended. In this position, the valve spool 58h opens the vent pathway 84 and blocks off the probe port 82. With the vent pathway 84 open, atmospheric air 76 flows freely into the valve spool passageway 86 to negate the vacuum that might otherwise exist in the valve spool passageway 86. Blocking the probe port 82 and venting the valve spool passageway 86 inhibits vacuum from being applied to the suction tube 35 in the flexible tubular probe 14.

Manually depressing 90 the valve spool 58h, as shown in FIG. 26, resiliently compresses the spring portion 118 of the valve spool 58h. In this position, the valve spool 58h opens the probe port 82 and closes the vent pathway 84. With the vent pathway 84 closed, atmospheric air 76 can no longer flow into the valve spool passageway 86, so the source of vacuum 52 can restore the vacuum in the valve spool passageway 86. The now open probe port 82 connects the vacuum in the valve spool passageway 86 to the suction tube 35 in the flexible tubular probe 14, delivering suction to the tip 45 of the suction tube 35.

In some examples, the suction valve 15h has extra radial clearance between the outer diameter of the valve spool 58h and the inner diameter of the valve housing 56h. The extra clearance provides room for the spring portion 118 to expand radially inward when compressed.

Upon manually releasing the pushbutton head 60, the resiliently compressed spring portion 118 relaxes and retracts. This returns the valve spool 58h to its home position and the suction valve 15h to its vented configuration.

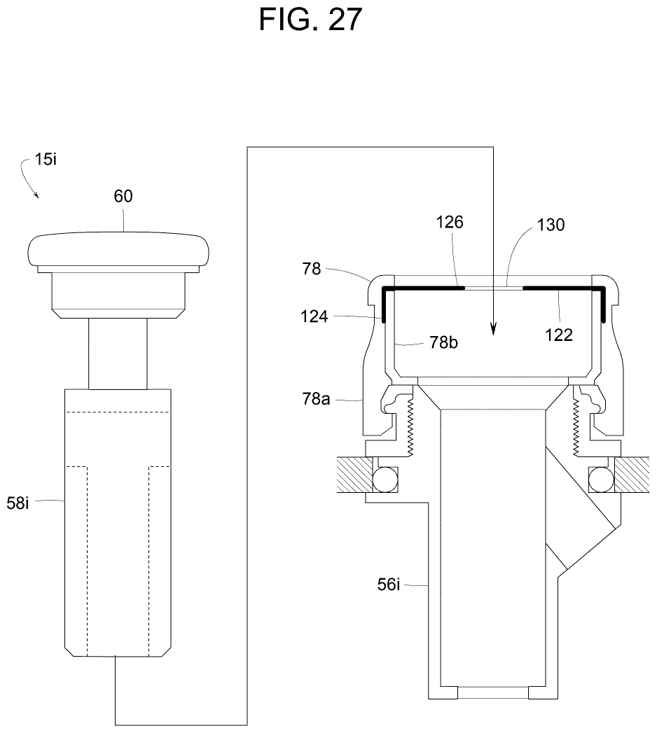

FIGS. 27-32 show suction valve 15i comprising the valve housing 56i and the valve spool 58i. FIG. 27 shows the valve spool 58i being readily inserted into the valve housing 56i without having to use tools. In this example, suction valve 15c includes a resiliently flexible diaphragm 122. In some examples, the diaphragm 122 is made of an elastomer (e.g., TPE or thermoplastic elastomer, silicone, polyurethane, neoprene, natural rubber, latex, synthetic rubber, elastane, etc.). In some examples, the diaphragm 122 is made of spring steel.

The diaphragm 122 has an outer periphery 124 and an inner periphery 126. In some examples, the outer periphery 124 is anchored to the valve housing 56i and/or to the handle body 20. In some examples, the outer periphery 124 is clamped or otherwise secured between an outer shell 78a and an inner liner 78b of the button guard 78. In some examples, the button guard 78 and the valve spool 58 define an annular gap 128 therebetween, and the diaphragm 122 extends in a radial direction across the annular gap 128. FIGS. 27-29 illustrate how the valve spool 58i can be selectively removed completely from and insertable fully into the valve housing 56i while the outer periphery 124 of the diaphragm 122 remains anchored to the valve housing 56i and/or the handle body 20.

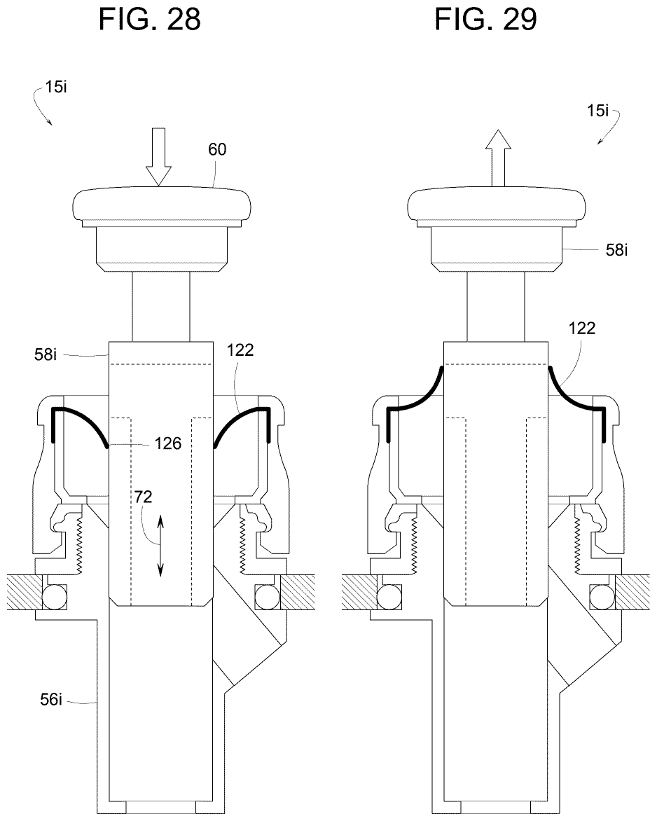

The diaphragm's inner periphery 126 defines an aperture 130. In some examples, the valve spool 58i extends in the lengthwise direction 72 through the aperture 130 to engage the inner periphery 126. In some examples, the diaphragm's elasticity allows the valve spool 58i to be readily inserted (FIG. 28) and removed (FIG. 29) even if the aperture 130 is smaller than the outer diameter of the valve spool 58i. In some examples, a radial press fit exists between the valve spool 58 and the diaphragm's inner periphery 126.

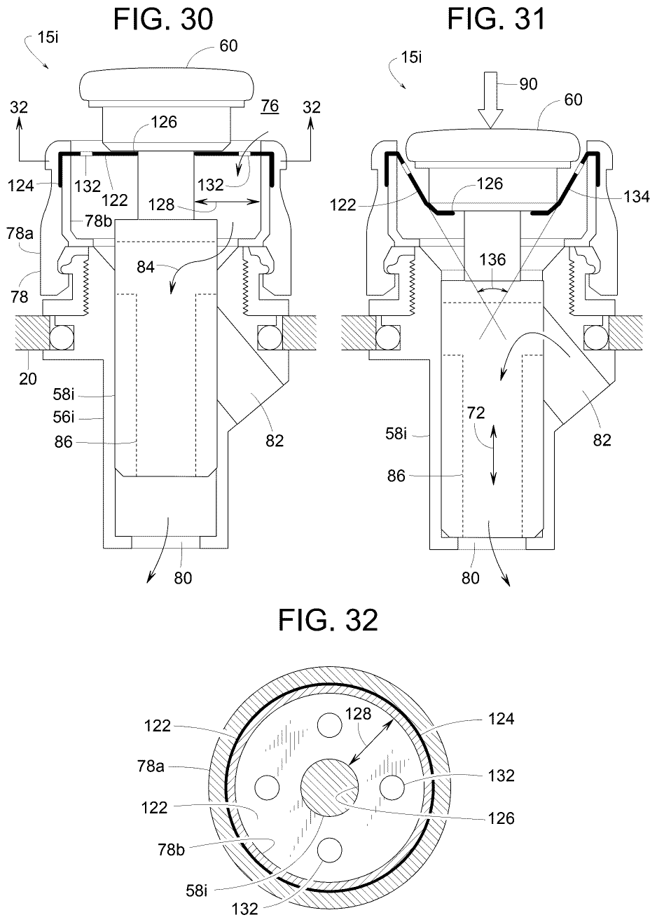

Moving valve spool 58i from a home position to a depressed position changes the configuration of the suction valve 15i from a vented configuration to a suction configuration. FIG. 30 shows the valve spool 58i at its home position to place the suction valve 15i in the vented configuration. FIG. 31 shows valve spool 58i at its depressed position, placing suction valve 15i in the suction configuration. The diaphragm 122, being resiliently flexible, urges the valve spool 58i to the home position.

In the vented configuration, shown in FIG. 30, the valve spool 58i opens the vent pathway 84 and blocks off the probe port 82. In some examples, the vent pathway 84 extends through one or more vent openings 132 in the diaphragm 122, as shown in FIG. 32. With the vent pathway 84 open, atmospheric air 76 flows freely into the valve spool passageway 86 to negate the vacuum that might otherwise exist in the valve spool passageway 86. Blocking the probe port 82 and venting the valve spool passageway 86 inhibits vacuum from being applied to the suction tube 35 in the flexible tubular probe 14.

Manually depressing 90 the valve spool 58i, as shown in FIG. 31, deflects the diaphragm 122, opens the probe port 82, and closes the vent pathway 84. In some examples, the diaphragm 122 comprises a conical surface 134 having a greater apex angle 136 when the valve spool 58i is in home position (FIG. 30) than in the depressed position (FIG. 31). In some examples, the apex angle 136 is approximately 180 degrees in the home position and about sixty degrees in the depressed position. This change in the apex angle 136 helps create a force component in the axial lengthwise direction 72 to urge the valve spool 58i to its home position.

In the depressed position, as shown in FIG. 31, the valve spool 58i blocks the vent pathway 84, so atmospheric air 76 can no longer flow into the valve spool passageway 86. This allows the source of vacuum 52 to restore the vacuum in the valve spool passageway 86. The now open probe port 82 connects the vacuum in the valve spool passageway 86 to the suction tube 35 in the flexible tubular probe 14, delivering suction to the tip 45 of the suction tube 35. Upon manually releasing the pushbutton head 60, as shown in FIG. 30, the diaphragm 122 pushes the valve spool 58i back up to its home position, which returns the suction valve 15i to its vented configuration.

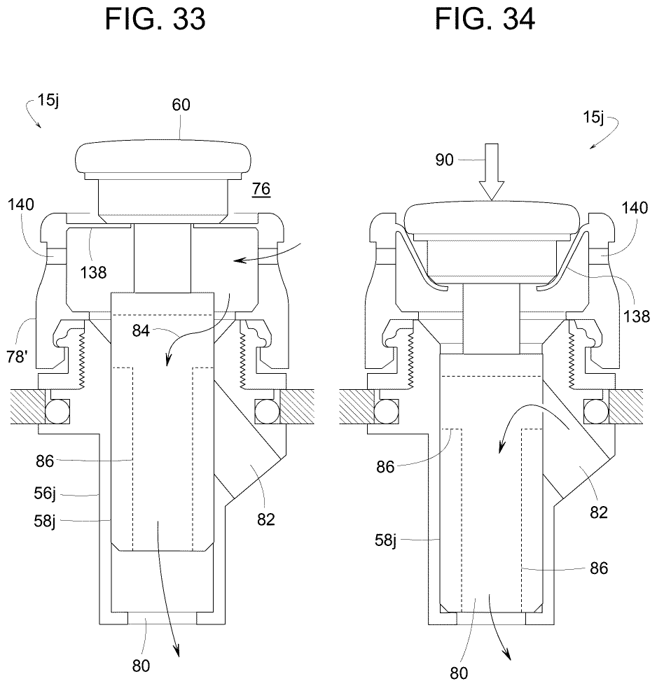

In the example shown in FIGS. 33 and 34, the suction valve 15j is similar to suction valve 15i, wherein FIGS. 33 and 34 correspond to FIGS. 30 and 31, respectively. The suction valve 15j comprises the valve housing 56j, the valve spool 58j, a button guard 78′, and a diaphragm 138. FIG. 33 shows the valve spool 58j in the home position to place the suction valve 15j in the vented configuration. FIG. 34 shows the valve spool 58j in the depressed position, placing the suction valve 15j in the suction configuration.

The diaphragm 138 of the suction valve 15j is similar in function to the diaphragm 122 of the suction valve 15i. In the example of the suction valve 15j, however, the diaphragm 138 is a seamless integral extension of the button guard 78′ to minimize the number of parts and simplify assembly during manufacturing. In some examples, the button guard 78′ and the diaphragm 138 are comprised of an elastomer (e.g., TPE or thermoplastic elastomer, silicone, polyurethane, neoprene, natural rubber, latex, synthetic rubber, elastane, etc.).

In addition or as an alternative to the vent openings 132, some examples of the suction valve 15j include one or more vent openings 140 through the button guard 78′. The vent openings 140 ensure that the vent pathway 84 is open when the suction valve 15j is in the vented configuration, as shown in FIG. 33.

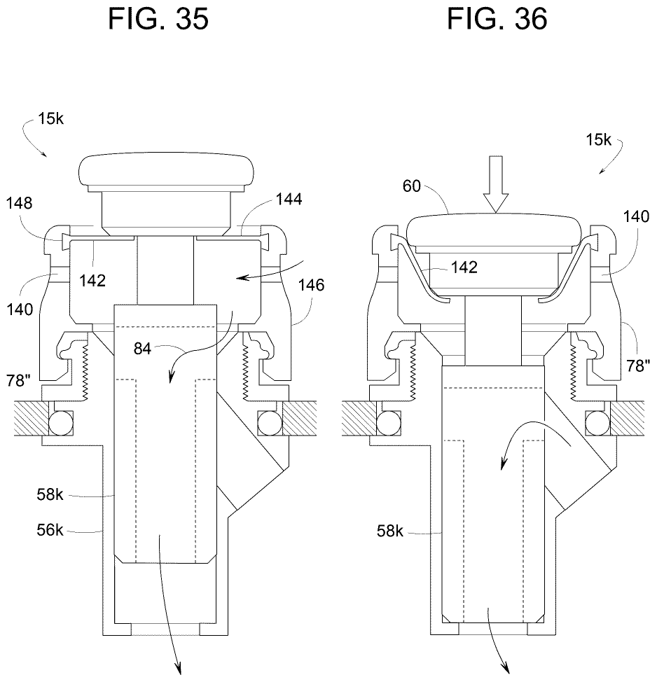

In the example shown in FIGS. 35 and 36, the suction valve 15k is similar to suction valve 15i, wherein FIGS. 35 and 36 correspond to FIGS. 30 and 31, respectively. The suction valve 15k comprises the valve housing 56k, the valve spool 58k, a button guard 78″, and a diaphragm 142. FIG. 35 shows the valve spool 58k in the home position to place the suction valve 15k in the vented configuration. FIG. 36 shows the valve spool 58k in the depressed position, placing the suction valve 15k in the suction configuration.

The diaphragm 142 of the suction valve 15k is similar in function to the diaphragm 122 of the suction valve 15i. In some examples of the suction valve 15k, however, the diaphragm 142 is an overmolded extension 144 of a main portion 146 of the button guard 78″ to create a single structure that, in some examples, includes a secure seam 148 between the diaphragm 142 and the button guard's main portion 146. In some examples, the diaphragm 142 is softer and more flexible than the main portion 146. In some examples, the diaphragm 142 is comprised of an elastomer (e.g., TPE or thermoplastic elastomer, silicone, polyurethane, neoprene, natural rubber, latex, synthetic rubber, elastane, etc.).

In addition or as an alternative to the vent openings 132, some examples of the suction valve 15k include one or more vent openings 140 through the button guard 78″. The vent openings 140 ensure that the vent pathway 84 is open when the suction valve 15k is in the vented configuration, as shown in FIG. 35.

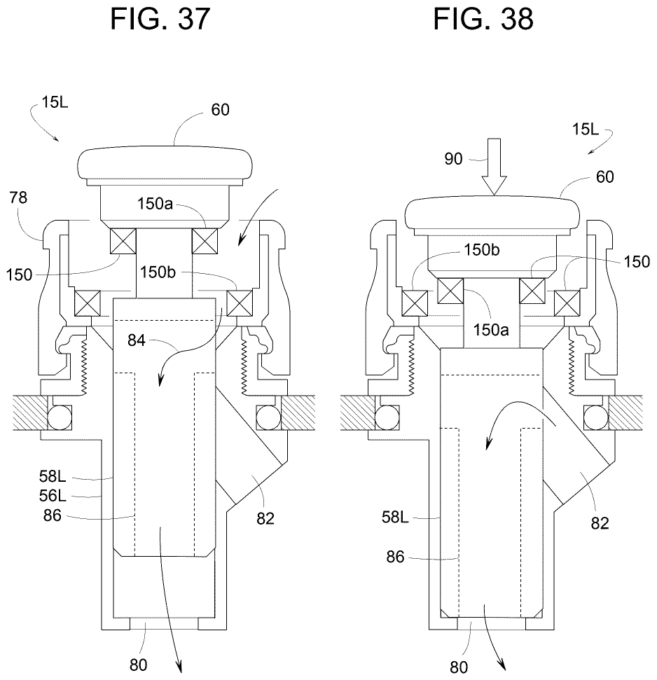

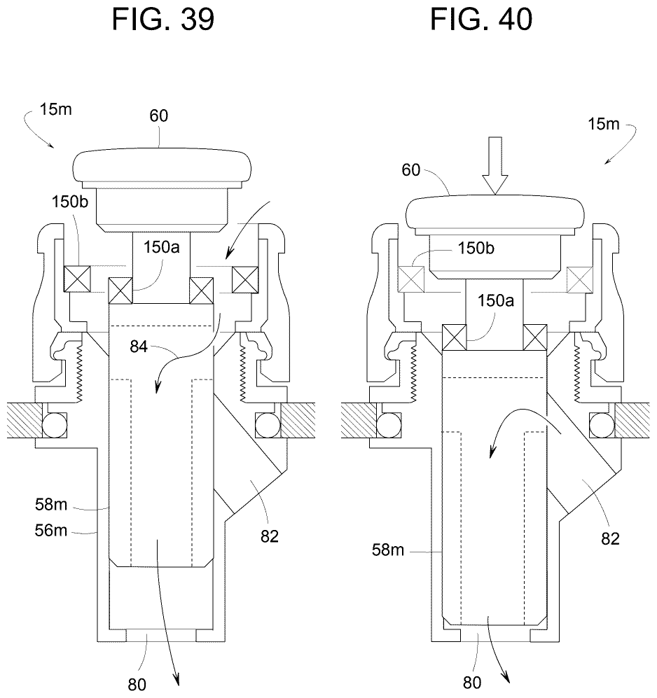

FIGS. 37 and 38 show the suction valve 15l comprising the valve housing 56l, the valve spool 58l, and a magnetic set 150. FIG. 37 shows the valve spool 58l in the home position to place the suction valve 15l in the vented configuration. FIG. 38 shows the valve spool 58l in the depressed position, placing the suction valve 15l in the suction configuration. In this example, the magnetic set 150 comprises a first ferromagnetic member 150a and a second ferromagnetic member 150b. In some examples, the ferromagnetic members 150a and 150b are magnetic (i.e., magnetized) with their magnetic poles arranged so as to repel each other, urging the valve spool 58l to the home position.

The term, “ferromagnetic” refers to a member that can be attracted or repelled by a magnetic field. Magnets and nonmagnetized iron are examples of ferromagnetic members. The terms, “magnet” and “magnetic” refer to ferromagnetic members that have been magnetized. The term, “magnetized” refers to a member that has been conditioned to have a magnetic field. The term, “magnetic set” refers to at least two members that are attracted or repelled by a magnetic field between the members.

In some examples, first ferromagnetic member 150a is attached to the pushbutton head 60 or to some other suitable part of the valve spool 58l. In some examples, the second ferromagnetic member 150b is attached to the button guard 78 or to some other part of the valve housing 56l. In some examples, each ferromagnetic member 150a and 150b is generally toroidal to encircle the valve spool 58l.

In some examples, as shown in FIGS. 39 and 40, the suction valve 15m comprises the valve spool 58m and the valve housing 56m. In this example, both of the ferromagnetic members 150a and 150b are magnetic (i.e., magnetized) with their magnetic poles arranged so as to attract each other, urging the valve spool 58l to the home position. FIG. 39 shows the valve spool 58m in the home position to place the suction valve 15m in the vented configuration. FIG. 40 shows the valve spool 58m in the depressed position, placing the suction valve 15m in the suction configuration.

Still referring to FIGS. 39 and 40, in some examples, only one of the ferromagnetic member 150a or 150b is magnetic (magnetized), while the other ferromagnetic member 150a or 150b is not magnetized but is still magnetically attracted, nonetheless. So the two ferromagnetic members 150a and 150b can still urge the valve spool 58l to the home position even though only one of the ferromagnetic members 150a and 150b is magnetized.

In some examples, the valve spool 58m is made of metal (e.g., stainless steel, brass, bronze, titanium, aluminum, etc.) while the valve housing 56m is made of a polymer such as, for example, nylon, PEEK (polyetheretherketone), polycarbonate, ABS (acrylonitrile-butadiene-styrene), HDPE (high-density polyethylene), UHMW (ultra-high molecular-weight polyethylene), POM (polyoxymethylene, polyacetal, Delrin, Celcon, etc.), POM-C (polyoxymethylene copolymer), and POM-H (polyoxymethylene homopolymer). Such a combination of metal and polymeric materials can provide an accurate fit and low friction between the valve spool 58 and the valve housing 56. In some examples, the valve housing 56 is a seamless integral part of the handle body 20.

Some examples of the suction valve 15a-m can be defined as described in the following examples 1-28.

Example-1 A suction valve for use in an endoscope that includes a handle body, the suction valve being configurable selectively to a suction configuration and a vented configuration, the suction valve comprising: a valve housing to be supported by the handle body; and a valve spool being movable between a home position and a depressed position relative to the valve housing, the suction valve being in the suction configuration when the valve spool is in the depressed position, the suction valve being in the vented configuration when the valve spool is in the home position, the valve spool extending in a lengthwise direction farther into the valve housing when the valve spool is in the depressed position than in the home position, at least one of the valve housing and the valve spool comprising both a spring portion and a rigid portion, the spring portion urging the valve spool to the home position.

Example-2 The suction valve of Example-1, wherein the spring portion and the rigid portion are comprised of a seamless unitary piece.

Example-3 The suction valve of Example-1, wherein the spring portion defines a plurality of slits distributed along the lengthwise direction.

Example-4 The suction valve of Example-1, wherein the spring portion defines a plurality of slits distributed along the lengthwise direction, and the plurality of slits are staggered circumferentially around the valve spool.

Example-5 The suction valve of Example-1, wherein the spring portion is a helical coil extending integrally and seamlessly from the rigid portion.

Example-6 The suction valve of Example-1, wherein the spring portion is softer than the rigid portion.

Example-7 The suction valve of Example-1, wherein the valve housing includes the spring portion and the rigid portion.

Example-8 The suction valve of Example-1, wherein the valve spool includes the spring portion and the rigid portion.

Example-9 The suction valve of Example-1, wherein the endoscope include a flexible tubular probe extending from the handle body and containing a suction tube, the suction valve further comprising: a suction source opening at least partially defined by at least one of the valve housing and the valve spool, the suction source opening being connected in fluid communication with a source of vacuum; a probe port at least partially defined by the valve housing, the probe port connecting the suction valve in fluid communication with the suction tube; a vent pathway at least partially defined by at least one of the valve housing and the valve spool; and a valve spool passageway at least partially defined by the valve spool and being in fluid communication with the suction source opening, the valve spool passageway connecting the suction source opening in fluid communication with probe port when the suction valve is in the suction configuration, the valve spool passageway connecting the suction source opening in fluid communication with the vent pathway when the suction valve is in the vented configuration.

Example-10 The suction valve of Example-9, further comprising a button guard anchored to the handle body and encircling the valve spool, wherein the button guard and the valve spool define an annular gap therebetween, and the annular gap is part of the vent pathway.

Example-11 The suction valve of Example-9, wherein the valve spool includes a pushbutton head, and the suction source opening and the pushbutton head are at opposite ends of the valve spool.

Example-12 The suction valve of Example-11, wherein the spring portion is closer to the suction source opening than to the pushbutton head.

Example-13 The suction valve of Example-11, wherein the probe port is at an intermediate position between the pushbutton head and the suction source opening, wherein the intermediate position between the pushbutton head and the suction source opening is with reference to the lengthwise direction.

Example-14 A suction valve for use in an endoscope that includes a handle body, the suction valve being configurable selectively to a suction configuration and a vented configuration, the suction valve comprising: a valve housing to be supported by the handle body; a diaphragm having an outer periphery and an inner periphery, the outer periphery being anchored to at least one of the valve housing and the handle body, the inner periphery defining an aperture; and a valve spool engaging the inner periphery of the diaphragm and extending in a lengthwise direction through the aperture and into the valve housing, the valve spool being movable between a home position and a depressed position relative to the valve housing, the suction valve being in the suction configuration when the valve spool is in the depressed position, the suction valve being in the vented configuration when the valve spool is in the home position, the valve spool extending farther into the valve housing when the valve spool is in the depressed position than in the home position, the diaphragm being resiliently flexible to urge the valve spool to the home position.

Example-15 The suction valve of Example-14, wherein the diaphragm comprises an elastomer.

Example-16 The suction valve of Example-14, further comprising a button guard anchored to the handle body and encircling the valve spool, wherein the button guard and the valve spool define an annular gap therebetween, and the diaphragm extends in a radial direction across the annular gap.

Example-17 The suction valve of Example-16, wherein the diaphragm is a seamless integral extension of the button guard.

Example-18 The suction valve of Example-16, wherein the diaphragm is an overmolded extension of a main portion of the button guard, and the diaphragm is softer than the main portion.

Example-19 The suction valve of Example-14, wherein the diaphragm comprises a conical surface having a greater apex angle when the valve spool is in home position than in the depressed position.

Example-20 The suction valve of Example-14, wherein the diaphragm at least partially defines a vent opening therethrough.

Example-21 The suction valve of Example-14, wherein the endoscope include a flexible tubular probe extending from the handle body and containing a suction tube, the suction valve further comprising: a suction source opening at least partially defined by at least one of the valve housing and the valve spool, the suction source opening being connected in fluid communication with a source of vacuum; a probe port at least partially defined by the valve housing, the probe port connecting the suction valve in fluid communication with the suction tube; a vent pathway at least partially defined by at least one of the valve housing and the valve spool; and a valve spool passageway at least partially defined by the valve spool and being in fluid communication with the suction source opening, the valve spool passageway connecting the suction source opening in fluid communication with probe port when the suction valve is in the suction configuration, the valve spool passageway connecting the suction source opening in fluid communication with the vent pathway when the suction valve is in the vented configuration.

Example-22 The suction valve of Example-14, wherein the valve spool is selectively removable completely from and insertable fully into the valve housing while the outer periphery of the diaphragm remains anchored to at least one of the valve housing and the handle body.

Example-23 The suction valve of Example-14, wherein a radial press fit exists between the valve spool and the inner periphery of the diaphragm.

Example-24 A suction valve for use in an endoscope that includes a handle body, the suction valve being configurable selectively to a suction configuration and a vented configuration, the suction valve comprising: a valve housing to be supported by the handle body; a valve spool being movable between a home position and a depressed position relative to the valve housing, the suction valve being in the suction configuration when the valve spool is in the depressed position, the suction valve being in the vented configuration when the valve spool is in the home position, the valve spool extending in a lengthwise direction farther into the valve housing when the valve spool is in the depressed position than in the home position; and a magnetic set comprising a first member substantially stationary relative to the valve spool and a second member substantially stationary relative to the valve housing, the magnetic set providing a magnetic force urging the valve spool to the home position.

Example-25 The suction valve of Example-24, wherein the first member is a first magnet, and the second member is a second magnet.

Example-26 The suction valve of Example-24, wherein only one member of the magnetic set is magnetized.

Example-27 The suction valve of Example-24, wherein the first member and the second member encircle the valve spool.

Example-28 The suction valve of Example-24, wherein the endoscope include a flexible tubular probe extending from the handle body and containing a suction tube, the suction valve further comprising: a suction source opening at least partially defined by at least one of the valve housing and the valve spool, the suction source opening being connected in fluid communication with a source of vacuum; a probe port at least partially defined by the valve housing, the probe port connecting the suction valve in fluid communication with the suction tube; a vent pathway at least partially defined by at least one of the valve housing and the valve spool; and a valve spool passageway at least partially defined by the valve spool and being in fluid communication with the suction source opening, the valve spool passageway connecting the suction source opening in fluid communication with probe port when the suction valve is in the suction configuration, the valve spool passageway connecting the suction source opening in fluid communication with the vent pathway when the suction valve is in the vented configuration.

The disclosure should not be considered limited to the particular examples described above. Various modifications, equivalent processes, as well as numerous structures to which the disclosure can be applicable will be readily apparent to those of ordinary skill in the art upon review of the instant specification.

1. A suction valve for use in an endoscope that includes a handle body, the suction valve being configurable selectively to a suction configuration and a vented configuration, the suction valve comprising:
a valve housing to be supported by the handle body; and
a valve spool being movable between a home position and a depressed position relative to the valve housing, the suction valve being in the suction configuration when the valve spool is in the depressed position, the suction valve being in the vented configuration when the valve spool is in the home position, the valve spool extending in a lengthwise direction farther into the valve housing when the valve spool is in the depressed position than in the home position, at least one of the valve housing and the valve spool comprising both a spring portion and a rigid portion, the spring portion urging the valve spool to the home position.

2. The suction valve of claim 1, wherein the spring portion and the rigid portion are comprised of a seamless unitary piece.

3. The suction valve of claim 1, wherein the spring portion defines a plurality of slits distributed along the lengthwise direction.

4. The suction valve of claim 1, wherein the spring portion defines a plurality of slits distributed along the lengthwise direction, and the plurality of slits are staggered circumferentially around the valve spool.

5. The suction valve of claim 1, wherein the spring portion is a helical coil extending integrally and seamlessly from the rigid portion.

6. The suction valve of claim 1, wherein the spring portion is softer than the rigid portion, wherein the valve housing includes the spring portion and the rigid portion, or wherein the valve spool includes the spring portion and the rigid portion.

7. The suction valve of claim 1, wherein the endoscope include a flexible tubular probe extending from the handle body and containing a suction tube, the suction valve further comprising:
a suction source opening at least partially defined by at least one of the valve housing and the valve spool, the suction source opening being connected in fluid communication with a source of vacuum;
a probe port at least partially defined by the valve housing, the probe port connecting the suction valve in fluid communication with the suction tube;
a vent pathway at least partially defined by at least one of the valve housing and the valve spool; and
a valve spool passageway at least partially defined by the valve spool and being in fluid communication with the suction source opening, the valve spool passageway connecting the suction source opening in fluid communication with probe port when the suction valve is in the suction configuration, the valve spool passageway connecting the suction source opening in fluid communication with the vent pathway when the suction valve is in the vented configuration.

8. A suction valve for use in an endoscope that includes a handle body, the suction valve being configurable selectively to a suction configuration and a vented configuration, the suction valve comprising:
a valve housing to be supported by the handle body;
a diaphragm having an outer periphery and an inner periphery, the outer periphery being anchored to at least one of the valve housing and the handle body, the inner periphery defining an aperture; and
a valve spool engaging the inner periphery of the diaphragm and extending in a lengthwise direction through the aperture and into the valve housing, the valve spool being movable between a home position and a depressed position relative to the valve housing, the suction valve being in the suction configuration when the valve spool is in the depressed position, the suction valve being in the vented configuration when the valve spool is in the home position, the valve spool extending farther into the valve housing when the valve spool is in the depressed position than in the home position, the diaphragm being resiliently flexible to urge the valve spool to the home position.

9. The suction valve of claim 8, wherein the diaphragm comprises an elastomer.

10. The suction valve of claim 8, further comprising a button guard anchored to the handle body and encircling the valve spool, wherein the button guard and the valve spool define an annular gap therebetween, and the diaphragm extends in a radial direction across the annular gap.

11. The suction valve of claim 10, wherein the diaphragm is a seamless integral extension of the button guard.

12. The suction valve of claim 10, wherein the diaphragm is an overmolded extension of a main portion of the button guard, and the diaphragm is softer than the main portion, wherein the diaphragm comprises a conical surface having a greater apex angle when the valve spool is in home position than in the depressed position, wherein the diaphragm at least partially defines a vent opening therethrough.

13. The suction valve of claim 8, wherein the endoscope include a flexible tubular probe extending from the handle body and containing a suction tube, the suction valve further comprising:
a suction source opening at least partially defined by at least one of the valve housing and the valve spool, the suction source opening being connected in fluid communication with a source of vacuum;
a probe port at least partially defined by the valve housing, the probe port connecting the suction valve in fluid communication with the suction tube;
a vent pathway at least partially defined by at least one of the valve housing and the valve spool; and
a valve spool passageway at least partially defined by the valve spool and being in fluid communication with the suction source opening, the valve spool passageway connecting the suction source opening in fluid communication with probe port when the suction valve is in the suction configuration, the valve spool passageway connecting the suction source opening in fluid communication with the vent pathway when the suction valve is in the vented configuration.

14. The suction valve of claim 8, wherein the valve spool is selectively removable completely from and insertable fully into the valve housing while the outer periphery of the diaphragm remains anchored to at least one of the valve housing and the handle body.

15. The suction valve of claim 8, wherein a radial press fit exists between the valve spool and the inner periphery of the diaphragm.

16. A suction valve for use in an endoscope that includes a handle body, the suction valve being configurable selectively to a suction configuration and a vented configuration, the suction valve comprising:
a valve housing to be supported by the handle body;
a valve spool being movable between a home position and a depressed position relative to the valve housing, the suction valve being in the suction configuration when the valve spool is in the depressed position, the suction valve being in the vented configuration when the valve spool is in the home position, the valve spool extending in a lengthwise direction farther into the valve housing when the valve spool is in the depressed position than in the home position; and
a magnetic set comprising a first member substantially stationary relative to the valve spool and a second member substantially stationary relative to the valve housing, the magnetic set providing a magnetic force urging the valve spool to the home position.

17. The suction valve of claim 16, wherein the first member is a first magnet, and the second member is a second magnet.

18. The suction valve of claim 16, wherein only one member of the magnetic set is magnetized.

19. The suction valve of claim 16, wherein the first member and the second member encircle the valve spool.

20. The suction valve of claim 16, wherein the endoscope include a flexible tubular probe extending from the handle body and containing a suction tube, the suction valve further comprising:
a suction source opening at least partially defined by at least one of the valve housing and the valve spool, the suction source opening being connected in fluid communication with a source of vacuum;
a probe port at least partially defined by the valve housing, the probe port connecting the suction valve in fluid communication with the suction tube;
a vent pathway at least partially defined by at least one of the valve housing and the valve spool; and
a valve spool passageway at least partially defined by the valve spool and being in fluid communication with the suction source opening, the valve spool passageway connecting the suction source opening in fluid communication with probe port when the suction valve is in the suction configuration, the valve spool passageway connecting the suction source opening in fluid communication with the vent pathway when the suction valve is in the vented configuration.
